(19) United States
(12) Patent Application Publication
(10) Pub. No.: US 2005/0192581 A1
(43) Pub. Date: Sep. 1, 2005

(54) Radiopaque, coaxial orthopedic tether design and method

(76) Inventors: Fred J. Molz, Collierville, TN (US); Troy D. Drewry, Memphis, TN (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/788,866

(22) Filed: Feb. 27, 2004

(51) Int. Cl. A61B 17/56

(52) U.S. Cl. 606/74

(57) ABSTRACT

This invention relates to orthopedic tethers and their use to treat orthopedic defects. The tethers of the present invention include a central or inner load bearing fiber or cable surrounded by one or more protective sheaths. Additionally the tethers of the present invention provide enhanced imaging characteristics and can include one or more a radiopaque elements that can readily observed under common diagnostic imaging techniques.

### BACKGROUND OF THE INVENTION

The present invention related to orthopedic devices for use in treating orthopedic defects. More specifically, the present invention is directed to orthopedic tethers to bind or secure bone and bone fragments together or to an ancillary orthopedic device; to methods of treating a patent with an orthopedic defect; and to methods of producing the orthopedic tether device.

Orthopedic defects are frequently treated by joining or securing the damaged or diseased bone portions together thereby allowing the bone to heal. The bones can be partly or fully immobilized to promote bone tissue growth or regeneration and/or healing of stretched or torn ligaments. Immobilization and or joining of the bone pieces is usually accomplished using a variety of bone plates, surgical cord, and some type of fastening device such as a screw, staple, or glue.

For articulating bone joints, such as the knees, hips and spinal column that have become damaged, bone plates alone may not be effective to either immobilize the bone pieces of the joint and/or support the adjoining bone portions. Consequently, surgical cord is frequently used either in place of or to augment the bone plates.

For spinal defects often a full or partial discectomy is performed. Typically, in this procedure a spacer and/or fusion-promoting implant is inserted into the prepared disc space. This may require that the affected vertebrae be distracted to allow sufficient clearance over or through the opposing cortical rims of the adjacent vertebrae to permit insertion the spacer or implant. After insertion, the vertebrae must be retracted using a surgical cord that has been attached to the spinal processes or to the vertebral bodies using bone fasteners.

However, current methodologies frequently use a single cable or braid of surgical cord to tension the bone portions or vertebrae. The single cord does not exhibit acceptable imaging characteristics under commonly used diagnostic imaging techniques, i.e., x-ray, fluoroscopy, CT, and MRI imaging techniques. The imaging characteristics of the cord are very important to ensure that the cord is properly placed, remains in its desired location, and is functioning as required to affect the desired treatment. Furthermore, for articulating joints the single cord can chafe against adjacent structures, whether those structures be adjacent bone structures or implanted devices such as bone plates, rods, screws, and the like. The chafing is undesirable because it weakens the surgical cord by cutting either part-way or completely through one or more of the filaments making up the cord. Furthermore, the frayed cord can irritate the surrounding tissue structure, which can be particularly painful for the patient.

In light of the above problems, the present invention provides a novel orthopedic tethering device that exhibits better imaging characteristics and/or resists fraying. The present invention also provides an advancement in the relevant field and provides a variety of additional benefits and advantages.

### BRIEF DESCRIPTION OF THE DRAWINGS

FIG. 1 is an illustration of one embodiment of an orthopedic tether in accordance with the present invention.

FIG. 2 is one embodiment of an orthopedic tether comprising a radiopaque strand incorporated into a layer of the tether in accordance with the present invention.

FIG. 3 is yet another embodiment of an orthopedic tether having a radiopaque filament in accordance with the present invention.

FIG. 4 is an illustration of a segment of a spinal column including three vertebrae secured using an orthopedic tether in accordance with the present invention.

FIG. 5 is an illustration of one embodiment of an orthopedic tether secured to a knee joint in accordance with the present invention.

### SUMMARY OF THE INVENTION

The present invention relates to an orthopedic tether or surgical cord and the manufacture and use thereof. Various aspects of the invention are novel, nonobvious, and provide various advantages. While the actual nature of the invention covered herein can only be determined with reference to the claims appended hereto, certain forms and features which are characteristic of the preferred embodiments disclosed herein are described briefly as follows.

In one form, the present invention provides a surgical tether for orthopedic treatment to secure to two adjacent bone portions. The orthopedic tether comprises: a cord or core having a tensile strength sufficient to maintain a desired distance or orientation of the two bone portions; a first sheath substantially encasing the cord, wherein the first sheath comprises a plurality of fibers and provides an abrasion resistant coating to the cord; a radiopaque element; and optionally, a second sheath. When the second sheath is present the second sheath substantially encases the first sheath and/or the cord. In preferred embodiments, the radiopaque element can include one or more radiopaque filaments that has been braided or otherwise attached to or integrated with one of the cord or either the first or second sheath.

In other forms the present invention provides a surgical tether for orthopedic treatment to secure to two adjacent bone portions. The surgical tether comprises: a cord having a tensile strength sufficient to maintain a desired distance or orientation of the two bone portions; a first sheath substantially encasing the cord, wherein the first sheath comprises a plurality of fibers and provides an abrasion resistant coating to the cord; and means for imparting enhanced image characteristics to the tether.

In still other forms, the present invention provides an orthopedic tether for orthopedic treatment to secure adjacent bone portions. The tether comprises: a cord having a tensile strength sufficient to maintain a desired distance or orientation of the bone portions; a first sheath substantially encasing the cord, said outer cord comprising a plurality of fibers; a radiopaque filament; and means for attaching the first sheath to the cord to provide an abrasion resistant coating to the cord.

In still yet other forms, the present invention provides a method for treating an orthopedic defect. The method comprises: securing a tether to a first bone portion, wherein the tether comprises a cord, a first sheath that substantially encases the cord, and a radiopaque element, such that the cord and the first sheath are free to move longitudinally relative to each other; and attaching the cord to a second bone portion to secure the first bone portion and the second bone portion at a desired distance or orientation relative to each other. The treatment can be used in conjunction with a wide variety of other treatment regimes including promotion of arthrodesis, treating fractured or displaced bone tissue, treatment of congenital defects, treatment of scoliosis or kyphosis, treatment of diseased or traumatized bone defects, and/or joint replacement.

Further objects, features, aspects, forms, advantages and benefits shall become apparent from the description and drawings contained herein.

### DETAILED DESCRIPTION OF THE INVENTION

For the purposes of promoting an understanding of the principles of the invention, reference will now be made to the embodiments illustrated herein, and specific language will be used to describe the same. It will nevertheless be understood that no limitation of the scope of the invention is thereby intended. Any alterations and further modifications in the described medical devices, surgical tethers, tether compositions, methods for treating patients, methods for preparing the devices, and any further applications of the principles of the invention as described herein are contemplated as would normally occur to one skilled in the art to which the invention relates.

The present invention generally relates to a surgical device that includes an orthopedic tether that provides advantageous properties to treat bone defects. The device can be used to treat a variety of bone defects including diseased, damaged, and/or fractured bone. The defective bone structures can be the result of damaged, traumatized, and/or diseased bone tissue. The present invention provides particularly advantages in the treatment of scoliosis and/or kyphosis. Furthermore, by use of the term “orthopedic device”, it is intended to include within its meaning a device or implant that can be used to treat or repair defective, diseased, and/or damaged tissue of the muscular/skeletal system(s) and can include attaching bone portions together, reinforcing a single unitary bone portion and/or attaching ligaments to one or more bone portions. Furthermore, the devices and methods described herein can be used to treat any type of bone or related tissue including, without limitation, articulating bone and bone joints, long bones, short bones, flat bones, cortical bone tissue, cancellous bone tissue and associated ligaments.

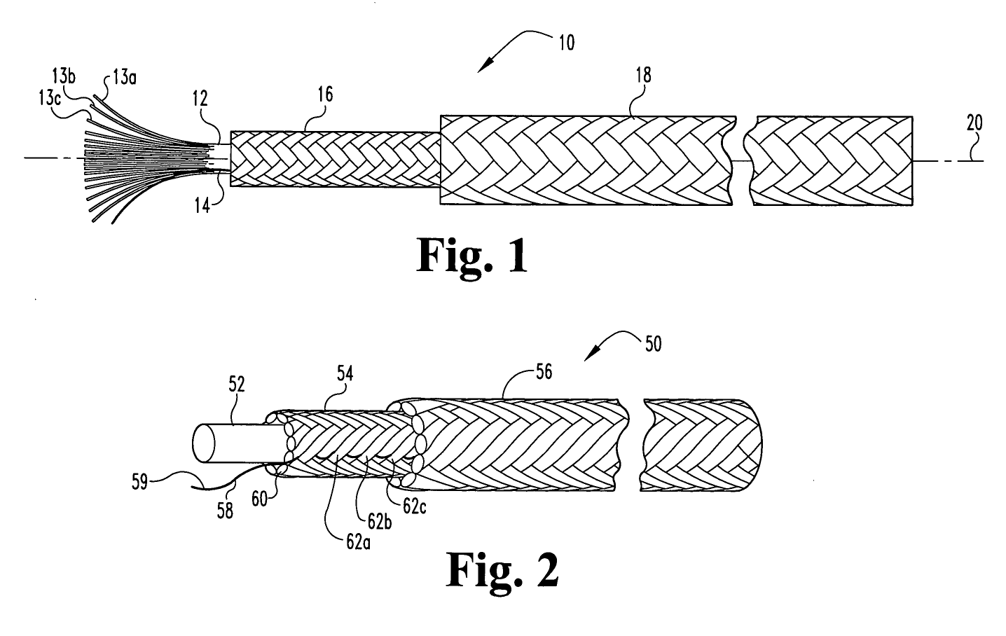

FIG. 1 is an illustration of one embodiment of an orthopedic tether 10 in accordance with the present invention. Tether 10 is illustrated as a coaxial tether having an inner cable or a cord 12 and at least one outer sheath or coating 18. Tether 10 is elongate, and consequently, defines a longitudinal axis 20. Furthermore, tether 10 can be flexible or rigid as desired.

In the illustrated embodiment, tether 10 includes a cord 12, which can be a single cord or core of material or a plurality or strands or filaments 13a, 13b, 13c . . . Cord 12 can be coated by at least one, and preferably more than one, exterior sheathing or coatings such as those illustrated as intermediate coating 16 and outer coating 18.

Inner cable or cord 12 can be formed from a variety of biocompatible or physiologically-acceptable materials including degradable and non-degradable polymeric materials, discussed more fully below. In one preferred embodiment, cord 12 is composed of a polymeric material such as a commercially available ultra high molecular weight polyethylene (UHMWPE).

The tethers of the present invention can be fabricated and/or composed of suitable material tailored to treat and repair a variety of muscular/skeletal defects and disorders. Physical characteristics and properties of the tether and associated components such as tensile strength, elasticity or stiffness and creep can be varied as desired. Tests measuring one or more of these properties can be based on ASTM D2990-95 “Standard Test Methods for Tensile, Compressive and Flexural Creep and Creep-Rupture of Plastics.

In one embodiment, cord 12 is provided to have a tensile strength sufficient to restrain or maintain the attached bone pieces or portions in a desired orientation and/or spacing with each other despite the biomechanical stresses exerted by the muscular/skeletal system during normal activity. In preferred embodiments, cord 12 is provided to have a tensile strength of at least about 500 N. In still more preferred embodiments, cord 12 is provided to have a tensile strength of greater than about 1,000 N; still more preferred to have a tensile strength greater than about 2000 N.

The elasticity or stiffness of the tethers can also be varied for a particular application or treatment. The stiffness of the tethers as used herein are defined as the load on the tether divided by the displacement or lengthening of the tether or cord under consideration. The stiffness is measured in units of Newtons per millimeter (N/mm). In one embodiment, the stiffness of the tethers of the present invention is about 1 N/mm or greater. In other embodiments the stiffness can be about 20 N/mm or greater; or about 150 N/mm; and still yet about 200 N/mm or greater. For selected applications it may be desirable fabricate a tether that exhibits a lower stiffness. Consequently, tethers in this embodiment are configured to exhibit a stiffness of less than about 250 N/mm, or alternatively less than about 100 N/mm.

In addition or in the alternative, tethers prepared according to the present invention can deform or creep under strain. For certain applications it may be desirable to limit that amount creep that the tether exhibits. In preferred embodiments when subjected to a stressed of 1000 N for 200 hours, the tethers can exhibit less than about 3.0% elongation, more preferably less than about 2.5% elongation, and still more preferably less than about 1.0% elongation. In yet other embodiment, the tethers can exhibit a creep of greater than about 5.0% elongation or greater than about 10% elongation when subjected to the stress conditions noted above.

It will be understood that when the tethers are composed of a plurality of filaments that are braided or woven together at least a portion of the tether's elongation can be attributed to the particular weave pattern and whether the filaments are loosely or tightly woven together. The values listed above are for a tightly woven tether. The tether's elongation can vary by as much as 1 to 5 times the above values for a loosely woven tether.

The cord can be provided in a variety of diameters. The cord can be substantially cylindrical or a flat, ribbon-like configuration, whether formed of a single filament or a plurality of filaments 13a, 13b, 13c . . . In preferred embodiments, the diameter of cord 12 is selected to be about 2-6 mm. When provided as a plurality of fibers, the fiber can be arranged and/or fashioned as desired including without limitation, braiding, wounding, parallel, twisting, and weaving (either 2 dimensional or 3 dimensional weaves).

In selected embodiments, cord 12 can be provided to exhibit suitable imaging characteristics including a specified radiopacity to enable the tether to be observed under common medical diagnostics imaging techniques. The radiopacity can help ascertain that the tether has been correctly placed, and remains in place, as desired. In one form, the radiopacity can be provided by incorporating a radiopaque element into cord 12. In the illustrated example, a radiopaque fiber or filament 14 is associated with cord 12. Filament 14 can be composed of a radiopaque material such as a metal filament or a polymeric filament that has been impregnated or coated with a radiopaque material such as a metallic material. Examples of radiopaque materials for use with the present invention are discussed below more fully.

Cord 12 can be covered by one or more outer coatings or sheaths. In the illustrated embodiment, cord 12 is substantially encased within an intermediate sheath or coating 16. Intermediate coating 16 can be provided as a braided sheath formed of a plurality of individual fibers or filaments. In one embodiment, intermediate coating 16 can be formed of materials similar to that described above for cord 12. In other embodiments, intermediate coating 16 can be formed of or comprises a material different than that used to form cord 12. In a particularly preferred embodiment, intermediate coating 16 can be formed of a polyester or PTFE composition.

Intermediate coating 16, substantially encases cord 12. However, intermediate coating 12 is not directly bonded, secured or adhered to the external surface of cord 12. Consequently, cord 12 can have either restricted movement or have free movement longitudinally within the interior of intermediate coating 16.

Either cord 12 or the coating 16 or both can be treated to increase the freedom of movement of one relative to the other, i.e., reduce the friction between the two. The treatment can include introducing a lubricating layer between cord 12 and coating(s) 16/18 or, alternatively, one or the other can include fibers or a material selected to increase the lubricity when compared with the cords/coating made without the fibers or material. For example the cord or coating can include fibers formed of nylon or PTFE or other fluorinated polymers that exhibit increased lubricity. The lubricating layer, fiber, or other material used with the cord and/or coating is selected to be biocompatible.

Optionally, tether 10 can include one or more outer coatings such as outer coating 18. Outer coating 18 can be formed of materials similar to that described above for intermediate coating 16 and/or cord 12. In one embodiment, outer coating 18 is provided of a material that is different from intermediate coating 16 and different from cord 12. In other embodiments, outer coating 18 is provided of a similar material either in composition, strength, and/or radiopacity as that provided by intermediate coating 16 or first coating 12.

Outer coating 18 can provide increased resistance to chafing and abrasion. Preferably outer coating 18 is composed of a material having a higher abrasion resistance than that used for first cord 12 and/or intermediate coating 16. In other forms, the higher abrasion resistance can be accomplished by varying the weave or braid configuration. In still yet other forms, the higher abrasion resistance can be a result of allowing one or more of the inner cords, such as cord 12 and/or intermediate coating 16, the freedom or restricted freedom to move within the interior of outer cord 18.

In the illustrated embodiment, outer coating 18 can be provided as a plurality of filaments or fibers. The filaments or fibers can be provided in the form of a braid, a weave, and/or spirally wound around intermediate coating 16. In other embodiments, outer coating 18 can be provided as a series of circular bands concentric about intermediate coating 16 and/or cord 12.

In use, outer coating 18 and/or intermediate coating 16 can provide a protective sheath to cord 12. This outer sheath inhibits fraying or chafing of the load-bearing cord 12, and thus protects cord 12 from degradation resulting from chafing against adjacent structures.

FIG. 2 is an illustration of an alternative embodiment of an orthopedic tether 50 in accordance with the present invention. Orthopedic tether 50 includes an inner core 52, an optional intermediate coating 54, and an outer coating 56. Tether 50 can be provided substantially as has been described above for tether 10, for example, cord 52 can be provided as either a single filament or a plurality of filaments.

Intermediate coating 54 can include a radiopaque marker or element 58. Radiopaque element 58 can be provided either as a coated fiber 59 or a radiopaque filament 60 exhibiting sufficient radiopacity to be readily observable under common diagnostic imaging techniques. In preferred embodiments, element 58 is provided as an elongate wire that has been woven into the mesh defined by the plurality of fibers 62a, 62b, 62c . . . Alternatively, radiopaque element 58 can be spirally wound, either around the exterior surface of intermediate coating 54 or between the intermediate coating 54 and outer coating 56. In still yet another alternative, radiopaque element 58 can be spirally wound about the inner surface of intermediate coating 54, exterior to the outer surface of cord 52.

Outer coating 56 substantially encases intermediate coating 54. However, in preferred embodiments, outer coating 56 is not directly bonded or secured to intermediate coating 54. Consequently, intermediate coating 54 is free to move or slide longitudinally within the interior of outer coating 56. This movement can be free—movement requiring little force to initiate the longitudinal movement. Alternatively, this movement can be restricted—primarily induced by the friction fit of the outer coating 56 about the exterior surface of intermediate coating 54.

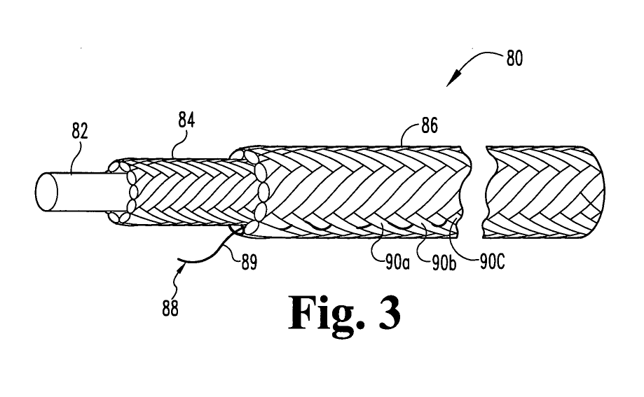

FIG. 3 is still yet another embodiment of an orthopedic tether 80 in accordance with the present invention. Tether 80 is comprised of a cord 82, intermediate coating 84, and outer coating 86. Tether 80 can be provided substantially as has been described above for tether 50 and/or tether 10.

Cord 82 can be provided as a single filament or fiber. Alternatively, cord 82 can be provided as a plurality of filaments or fibers, which can either extend substantially parallel with each other and/or be braided or woven together to form an integral cord. Cord 82 can be provided substantially as has been described above for cords 52 and 12.

Intermediate coating 84 can be provided substantially as has been described above for intermediate coating 16 with or without the inclusion of a radiopaque element.

Outer coating 86 can be provided to exhibit suitable or desirable imaging characteristics. These imaging characteristics can be accomplished by including within outer coating 86 a radiopaque element 88. Radiopaque element 88 can be provided substantially as has been described above for radiopaque element 58 and can include a ribbon or wire braid 89 within the individual filaments 90a, 90b, 90c . . . that compose outer coating 86. As before, radiopaque element 88 can be braided within the weaving or braids of outer coating 86. Alternatively, radiopaque element 88 can be spirally wound about or provided as concentric bands encasing outer coating 86.

In each of the above embodiments, tethers 10, 50, and 80 are illustrated as a coaxial tether including two or more cords or coatings. It will be understood that each of the individual cords or coatings can have substantially the same length. Alternatively, one or more of the intermediate or outer coatings can be truncated relative to the other coatings or cords. For example, referring specifically to tether 10 in FIG. 1, one or more of intermediate coatings 16 and/or outer coating 18 can be truncated to allow the underlying coatings/cords to extend beyond the truncated coating.

Preferably each of the individual cords or coatings are free to slide or move longitudinally relative to the other cords or coatings making up the tether. Consequently, in use the cord can be provided as a load-bearing or tensioning member. As such, the cord can be securely attached to one or more bone portions or fragments. The outer coatings may, but need not, be secured to the bone fragments or portions. Regardless one or more of the outer coatings can move in relation to the first coating. Consequently, when the outer coatings bear against adjacent structures the outer coatings provide a layer of protection for the cord. Additionally, when the outer coatings are not fixedly secured to the bone portions, the outer coatings can bear against the adjacent structures and remain engaged thereto and move with the adjacent structures or remain stationary with the adjacent structures regardless of whether the cord moves or not. This inhibits chafing or abrading of the outer coating and/or the cord.

The biodegradable material included in one or more of the cords, filaments, and/or matrices described above can be formed or composed of a variety of materials including, without limitation, degradable or resorbable polymeric materials, and composite materials.

The biodegradable materials for use in the present invention can include polymeric materials formed from oligomers, homopolymers, copolymers, and polymer blends that include polymerized monomers derived from l, d, or d/l lactide (lactic acid); glycolide (glycolic acid); ethers; amino acids; anhydrides; orthoesters; hydroxy esters; and mixtures of these monomeric repeating units.

Use of the term “copolymers” is intended to include within the scope of the invention polymers formed of two or more unique monomeric repeating units. Such copolymers can include random copolymers; graft copolymers; body copolymers; radial body, dibody, and tribody copolymers; alternating copolymers; and periodic copolymers. Use of the term “polymer blend” is intended to include polymer alloys, semi-interpenetrating polymer networks (SIPN), and interpenetrating polymer networks (IPN).

In a preferred embodiment, the biodegradable material comprises a biodegradable polymeric material including: poly(amino acids), polyanhydrides, polycaprolactones, poly(lactic-glycolic acid), polyhydroxybutyrates, polyorthoesters, and poly(d,l-lactide).

In still other embodiments, the biodegradable material can be formed of composite materials. Examples of composite materials include as a base material or matrix, without limitation: ceramics, resorbable cements, and/or biodegradable polymers listed above. Each of the base materials can be impregnated or interspersed with fibers, platelets, and/or particulate reinforcing materials.

A non-biodegradable or biostable materials for use in the present invention can include, without limitation; polymeric materials include polymerized monomers derived from: olefins, such as ethylene, propylene, butene-1, pentene-1, hexene-1,4-methylpentene-1, styrene, norbornene and the like; butadiene; polyfunctional monomers such as acrylate, methacrylate, methyl methacrylate; esters, for example, caprolactone and hydroxy esters; and mixtures of these monomeric repeating units.

The polymeric materials can also include: polyolefins, such as polyethylene, polypropylene, fluoropolymers, for example, polytetrafluoroethylene (PTFE), polyamides, polyethylene terephthalate (PET), polyesters, for example DACRON™ polyaramid, for example, KELVAR™, silicon rubbers, polyurethane, polyvinylchloride, carbon poly(ether, ether, ketone) (PEEK), poly(aryl ether, ketone) (PAEK), and the like.

Additionally the tethers of the present invention can be elastic. The tethers can be composed of elastic polymeric materials which can be either bidegradable or non biodegradable. Examples of suitable elastic materials for use in the present invention include: silicon rubbers. PEEK, nylons, poly(ethylene glycol) (PEG), polyolefins, polyurethanes, polycaprolactones, poly(lactic-glycolic acid), polyhydroxybutyrates, polyorthoesters, and poly(d,l-lactide) and the like. It will be understood that some of the materials listed above can exhibit variable properties depending upon the manner in which they are processed.

Preferred polymers for use in the present invention include ultra high molecular weight polyethylene, polyethylene, polyester, polypropylene and the like.

The radiopaque element can be provided in a variety of materials. Examples of radiolucent materials that can be used in the present invention include, without limitation: nitinol, titanium, titanium-vanadium-aluminum alloy, cobalt-chromium alloy, cobalt-chromium-molybdenum alloy, cobalt-nickel-chromium-molybdenum alloy, stainless steel, tantalum, niobium, hafnium, tungsten, gold, silver, platinum, or iridium metals, alloys, and mixtures thereof. In preferred embodiments, the radiopaque element is provided as a radiolucent metallic wire formed of one or more of the above listed materials. One particularly preferred material is a cobalt-chromium alloy sold under the trade name ELGILOY® by Elgiloy Specialty Metals of Elgin, Ill. (as specified in ASTM F1058). In other embodiments, the radiopaque element can be provided as a polymeric fiber(s) coated or impregnated with one or more of the materials listed above.

The tethers of the present invention can also exhibit suitable radiopacity by treating one or more of the cords, fibers, filaments, sheaths or coatings with a radiopacity inducing material such as barium sulfate. For example, the cord or the sheaths of any of tethers 10, 50 and 80, can be soaked in an aqueous solution of BaSO₄. This can introduce either long-term or short radiopaque markers into the treated tethers as desired.

The effective duration in vivo of the radiopaque marker can be varied as desired. The can be accomplished by a variety of methods including providing a radiopaque filament composed of a biodegradable material and soaking a filament with a solution of BaSO₄. The effective duration as used herein means the length of time in vivo that the radiopaque marker can be observed in vivo using common diagnostic imaging techniques. In practice, the effective duration can be selected to be between as short as one month and essentially indefinitely or for as long as the tether remains implanted within the patient. In other embodiments the effective duration of the radiopaque marker can be selected to be longer than about three month, more preferable longer than about six months, and still more preferably longer than about two years. As noted above, the maximum effective duration of the radiopaque marker can be essentially indefinitely. In other embodiments the effective duration of the radiopaque marker can be selected to be shorter than about 5 years or alternatively shorter than about 3 years.

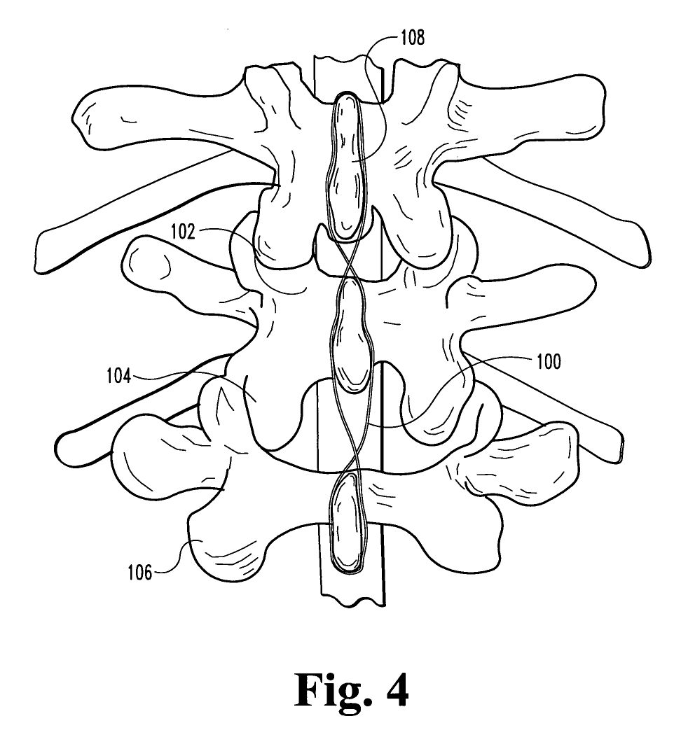

FIG. 4 is one embodiment of a tether 100 used to treat a bone defect such as that found in a spinal column between adjacent vertebrae or on a single vertebra. FIG. 4 illustrates the use of tether 100. Tether 100 can be provided substantially as has been described above for tethers 80, 50, and/or 10. Tether 100 is illustrated as an elongate flexible cable. Tether 100 can be of a suitable length to be secured to a number of vertebrae. Preferably tether 100 is provided in a length substantially longer than that need or desired to interconnect the vertebrae selected for treatment. In the procedure, tether 100 can be secured to one or more bone portions. The bone portions are illustrated as vertebra 102, vertebra 104, and vertebra 106. Tether 100 can be secured to one or more vertebrae by a variety of methods including tying around the specific portions of the bone such as spinal process 108 or by securing one or more portions of the tether with a variety of fasteners. The fasteners can include one or more of a screw, staple, glue, nail, bone hook, and the like. It will be understood that the tether need not be secured to each vertebrae. For example, tether 100 can be secured to vertebra 102 and vertebra 106, but not to vertebra 104. Examples of treatments that can be affected or advanced using the tethers of the present invention are also discussed in U.S. Pat. Nos. 6,616,669 and 6,299,613 both of which are incorporated by reference herein.

After tether 100 has been secured as desired to the selected vertebrae, any excess length or the ends of tether 100 can be removed. For example, the ends of tether 100 can be cut with any scalpel, surgical knife, scissors, laser, or cautery device commonly used in surgical procedures. In preferred embodiments, the ends of tether 100 are cut as desired to a selected length. Then the ends are sealed with heat with a cautery or laser. Heat sealing the ends of the implanted tether prevents fraying and disassembly of the tether.

The tethers of the present invention provide particular advantages in the treatment of scoliosis, through fusionless tethering. The correction of the deformity can be achieved by attaching the tether to the vertebral bodies on the convex side of the spine. The tether will minimize or arrest growth on the convex or “long” side of the spine and allow the concave or “short” side of the spine to grow and catch up with the long side. Alternatively, fusionless tethering may treat abnormal spinal alignment by simply preventing further misalignment such as curve progression. A wide variety of surgical approaches may be used in implementing tethering of the convex side. One approach is an open thoracotomy (standard). Another surgical approach contemplated is a minimally invasive thoracoscopic approach (endoscopic). The surgical approach may also be a combined anterior/posterior approach (standard or endoscopic). It should be understood that the invention can be practiced using other surgical approaches known to persons of ordinary skill in the art.

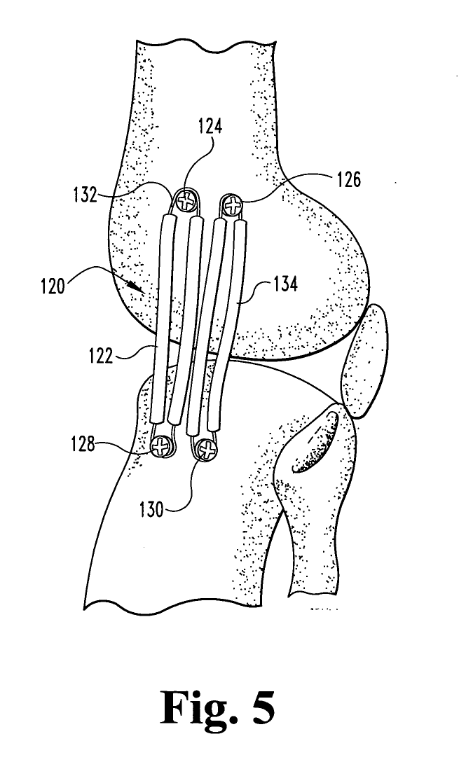

FIG. 5 is an illustration of a tether 120 that has been secured to an articulating knee joint. Tether 120 can be used to augment or replace one or more of the ligaments joining the bone in the knee joint. In the illustrated embodiment, tether 120 is provided as a single long cable 122 that has been attached at different locations on the femur and the tibia bones using a plurality of bone fasteners 124, 126, 128, and 130. Cable 122 is composed of an inner or cord 132 that is substantially encased within a sheath 134. Sheath 134 can be composed of one, two, three or more outer coatings, such as described above for intermediate coatings 16, 54, and 84 and/or outer coatings 18, 56, and 86. In the illustrated embodiment, sheath 134 is not secured to either the femur or the tibia. Further, it can be observed that at least a portion of cord 132 is exposed and not surrounded or encased within sheath 134.

One or more of tethers 10, 50, 80, 100, and/or 120 can be manufactured according to procedures known in the art. For example, a cord can be extruded either as a single filament or spirally wound as a plurality of parallel or braided filaments. Thereafter, one or more of the intermediate and/or outer coatings can be spirally wound around the pre-formed cord. In alternative embodiments, the tether can be provided and manufactured in a sequential operation which extrudes first the inner core material either as a single filament or a plurality of filaments either parallel or woven or braided together. Immediately thereafter, one or more of the intermediate and/or coating layers can be applied to the underlying coating or cord.

Examples of other orthopedic devices including cords and or rods that can be used in accordance with the present invention include those described in US patent application serial numbers, ser. No. 10/637,738, filed Aug. 8, 2003; and Ser. No. 10/442,821, filed May 21, 2003 and in U.S. Pat. Nos. 6,616,669; 6,436,099; and 6,299,613, all which are incorporated by reference herein.

The present invention contemplates modifications as would occur to those skilled in the art. It is also contemplated that tethers, cords, and materials, embodied in the present invention can be altered, substituted, combined, or added to as would occur to those skilled in the art without departing from the spirit of the present invention. In addition, the various treatment methods and manufacturing operations may be altered, rearranged, substituted, or combined as would occur to those skilled in the art. All publications, patents, and patent applications cited in this specification are herein incorporated by reference as if each individual publication, patent, or patent application was specifically and individually indicated to be incorporated by reference and set forth in its entirety herein.

Unless specifically identified to the contrary, all terms used herein are used to include their normal and customary terminology.

Further, while various embodiments of tethers and cords or filaments having specific components and structures are described and illustrated herein, it is to be understood that any selected embodiment can include one or more of the specific components and/or structures described for another embodiment where possible.

Further, any theory of operation, proof, or finding stated herein is meant to further enhance understanding of the present invention and is not intended to make the scope of the present invention dependent upon such theory, proof, or finding.

While the invention has been illustrated and described in detail in the drawings and foregoing description, the same is considered to be illustrative and not restrictive in character, it is understood that only the preferred embodiments have been shown and described and that all changes and modifications that come within the spirit of the invention are desired to be protected.

1. A surgical tether for orthopedic treatment to secure to two adjacent bone portions, said tether comprising:
a cord having a tensile strength sufficient to maintain a desired distance or orientation of the two bone portions;
a first sheath substantially encasing the cord, said first sheath comprising a plurality of fibers and providing an abrasion resistant coating to the cord;
a radiopaque element; and
optionally, a second sheath, said second sheath substantially encasing the first sheath.

2. The tether of claim 1 wherein the cord is slidably received within the second outer cord.

3. The tether of claim 1 wherein the cord is elongate and defines a longitudinal axis and wherein the cord is free to move longitudinally with respect to the first sheath.

4. The tether of claim 1 wherein the first and first sheaths are frictionally engaged with each other.

5. The tether of claim 1 wherein the cord consists of a single fiber.

6. The tether of claim 1 wherein the cord comprises a plurality of fibers.

7. The tether of claim 6 wherein the plurality of fibers are braided to provide the cord.

8. The tether of claim 7 wherein the radiopaque element comprises a single radiopaque filament woven in the plurality of fibers.

19. The tether of claim 1 wherein the plurality of fibers are braided to provide the first sheath.

10. The tether of claim 1 wherein the radiopaque element comprises barium sulfate.

11. The tether of claim 1 wherein the first sheath comprises a radiopaque element.

12. The tether of claim 1 wherein the radiopaque element comprises a single radiopaque filament woven in the plurality of filaments.

13. The tether of claim 1 wherein the radiopaque element comprises a plurality of radiopaque filaments.

14. The tether of claim 1 wherein the radiopaque element comprises one or more radiopaque filaments spirally wound around the first or first sheath.

15. The spinal tether of claim 1 comprising the optional second sheath substantially encasing the first sheath wherein second sheath is not fixedly secured to either the cord or the first sheath.

16. The tether of claim 15 wherein the second sheath comprises a plurality of braided fibers.

17. The tether of claim 15 wherein the radiopaque fiber is embedded within the second sheath.

18. The tether of claim 1 wherein cord is elongate and defines a longitudinal direction and the second sheath is free move longitudinally with respect to the first sheath or the cord.

19. The tether of claim 1 wherein the tether is attached to a plurality of bone portions.

20. The tether of claim 1 wherein the cord or the first sheath or both are composed of an elastomeric material.

21. The tether of claim 1 wherein the two bone portions include a first and second vertebrae.

22. The tether of claim 1 wherein the two bone portions include an articulating joint.

23. The tether of claim 1 wherein the cord and the first sheath are flexible.

24. The tether of claim 1 wherein the cord is composed of a polymeric material selected from the group consisting of: polyethylene, ultra high molecular weight polyethylene, polypropylene, fluoropolymers, polytetrafluoroethylene, polyamides, polyethylene terephthalate, polyesters, polyaramid, silicon rubbers, polyurethane, polyvinylchloride.

25. The tether of claim 24 wherein the first sheath is composed of a material different from the cord.

26. The tether of claim 25 wherein the first sheath is composed of a material selected from the group consisting of: polyethylene, polypropylene, fluoropolymers, polytetrafluoroethylene, polyamides, polyethylene terephthalate, polyesters, polyaramid, silicon rubbers, polyurethane, polyvinylchloride.

27. The tether of claim 1 wherein the cord and first sheath are composed of a biodegradable material.

28. The tether of claim 1 wherein the cord and first sheath are composed of a non-biodegradable material.

29. The tether of claim 1 comprising a first bone fastener and a second bone fastener to secure the tether to the two bone portions.

30. The tether of claim 29 wherein the first and second bone fasteners secure the cord to the first and second bone portions.

31. The tether of claim 30 wherein the first sheath is not secured to the two bone portions.

32. The tether of claim 30 comprising the second sheath and wherein the second sheath is not secured to the two or more bone portions.

33. The tether of claim 1 wherein the radiopaque element is composed of a biocompatible metallic fiber.

34. The tether of claim 33 wherein the radiopaque element is composed of a material selected from the group consisting of: nitinol, titanium, titanium-vanadium-aluminum alloy, cobalt-chromium alloy, cobalt-chromium-molybdenum alloy, cobalt-nickel-chromium-molybdenum alloy, stainless steel, tantalum, niobium, hafnium, tungsten, gold, silver, platinum, and iridium metals, alloys, and mixtures thereof.

35. The tether of claim 1 wherein the radiopaque element exhibits an effective duration in vivo of between about one month and about 5 years.

36. A surgical tether for orthopedic treatment to secure to two adjacent bone portions, said tether comprising:
a cord having a tensile strength sufficient to maintain a desired distance or orientation of the two bone portions;
a first sheath substantially encasing the cord, said first sheath comprising a plurality of fibers and providing an abrasion resistant coating to the cord; and
means for imparting radiolucency to the tether.

37. A surgical tether for orthopedic treatment to secure adjacent bone portions, said tether comprising:
a cord having a tensile strength sufficient to maintain a desired distance or orientation of the bone portions;
a first sheath substantially encasing the cord, said first sheath comprising a plurality of fibers;
a radiopaque filament engaged with either the cord or the first sheath; and
means for attaching the first sheath to the cord to provide an abrasion resistant coating to the cord.

38. A method for treating an orthopedic defect, said method comprising:
securing a tether to a first bone portion, said tether comprising a cord, a first sheath substantially encasing the cord, and a radiopaque element, wherein the cord and the first sheath are free to move longitudinally relative to each other; and
attaching the cord to a second bone portion to secure the first bone portion and the second bone portion at a desired distance or orientation relative to each other.

39. The method of claim 38 wherein said securing comprises securing the cord to the first bone portion.

40. The method of claim 38 wherein the tether comprises a second sheath.

41. The method of claim 38 wherein the radiopaque element comprises a radiopaque fiber attached to the second sheath.

42. The method of claim 38 wherein the radiopaque element comprises a radiopaque fiber attached to either the first or first sheath.

43. The method of claim 38 wherein the radiopaque element comprises a radiopaque fiber attached to the cord.

44. The method of claim 38 wherein the radiopaque element comprises a radiopaque fiber attached to the first sheath.

45. The method of claim 38 comprising positioning the first and second bone portions into a desired orientation or in close proximity to each other.

46. The method of claim 38 wherein the first and second bone portions are first and second vertebrae.

47. The method of claim 38 wherein the first and second bone portions each comprise a long bone.

48. The method of claim 38 wherein the first and second bone portions comprise an articulating joint.

49. The method of claim 38 wherein the tether is composed of one or more biodegradable materials.

50. The method of claim 38 wherein the tether is composed of a non-biodegradable material.

51. The method of claim 38 wherein the cord is composed of a material different from the first sheath.

52. The method of claim 38 wherein the cord is composed of a material selected from the group consisting of: polyethylene, ultra high molecular weight polyethylene, polypropylene, fluoropolymers, polytetrafluoroethylene, polyamides, polyethylene terephthalate, polyesters, polyaramid, silicon rubbers, polyurethane, polyvinylchloride.

53. The method of claim 38 wherein the first sheath is composed of a material selected from the group consisting of: polyethylene, ultra high molecular weight polyethylene, polypropylene, fluoropolymers, polytetrafluoroethylene, polyamides, polyethylene terephthalate, polyesters, polyaramid, silicon rubbers, polyurethane, polyvinylchloride.

54. The method of claim 38 wherein the radiopaque element is composed of a biocompatible metal fiber.

55. The method of claim 38 wherein the biocompatible metal fiber is selected from the group consisting of: nitinol, titanium, titanium-vanadium-aluminum alloy, cobalt-chromium alloy, cobalt-chromium-molybdenum alloy, cobalt-nickel-chromium-molybdenum alloy, stainless steel, tantalum, niobium, hafnium, tungsten, gold, silver, platinum, barium sulfate, and iridium metals, alloys, and mixtures thereof.

56. The method claim 38 wherein the tether is secured to more than two bone portions.

57. The method of claim 38 comprising cutting the tether to a desired length.

58. The method of claim 57 comprising heat sealing the cut ends of the tether.
